(19) United States
(12) Patent Application Publication
(10) Pub. No.: US 2018/0088119 A1
(43) Pub. Date: Mar. 29, 2018

(54) HIGH RESOLUTION ASSAYS FOR PROSTATE CANCER

(71) Applicants: PharmaSeq, Inc., Monmouth Junction, NJ (US); The Johns Hopkins University, Baltimore, MD (US)

(72) Inventors: Wlodek Mandecki, Princeton Junction, NJ (US); Ji Li, Hightstown, NJ (US); Zhen Yuan, Hillsborough, NJ (US); Robert William Veltri, Baldwin, MD (US); Christhunesa Soundararajan Christudass, Baltimore, MD (US)

(21) Appl. No.: 14/669,771

(22) Filed: Mar. 26, 2015

Related U.S. Application Data
(60) Provisional application No. 61/984,430, filed on Apr. 25, 2014.

(51) Int. Cl. G01N 33/574

(52) U.S. Cl. CPC G01N 33/57434; G01N 2333/495

(57) ABSTRACT

Provided in an embodiment is a high resolution method of detecting prostate cancer comprising utilizing a solid phase immunoassay to determine if a patient fluid shows a MIC-1 value in Zone M. In an embodiment, the method comprises conducting a sandwich assay in an assay device that, if the determined value is in Zone M, automatically generates a report stating that a high risk of prostate cancer exists.

Also provided in an embodiment a high resolution method of detecting prostate cancer comprising utilizing a solid phase immunoassay to determine if a patient serum shows a MIC-1 value and a PSA value in Zone A (defined below) or, if utilized, Zone B. In an embodiment, the method comprises conducting a sandwich assay in an assay device that, if the determined value is in Zone A and/or Zone B, automatically generates a report stating that a high risk of prostate cancer exists.

### CROSS-REFERENCE TO RELATED APPLICATION

This application claims benefit of U.S. Provisional Patent Application No. 61/984,430 filed Apr. 25, 2014, which is hereby incorporated in its entirety.

The present application relates generally to assays for prostate cancer, and devices for measuring such assays.

This invention was made with government support under an SBIR Phase I contract awarded by the National Institutes of Health awarded by the National Institutes of Health, Contract No. HSSN261201200069C. The government has certain rights in the invention.

Prostate cancer (PCa) is the most common malignancy among men in the United States, with 240,890 newly diagnosed cases and 33,720 deaths in 2011 (American 2011). Until now, a PSA test and a digital rectal examination (DRE) have been routinely used to screen for PCa in many countries and have been approved in the USA by the National Comprehensive Cancer Network (NCCN), American Urological Association (AUA), American Cancer Society (ACS), and the National Cancer Institute (NCI). PSA screening in the USA (Jemal et al 2010) has revolutionized the management of prostate cancer over the past two decades, especially with regards to early detection, greatly improving the chances of a curative treatment (Bastian et al. 2009). However, a new problem emerged over the years: overdiagnosis and overtreatment of PCa (Etzioni et al. 2002, Klotz 2010). This overdiagnosis is estimated to constitute about 56% of cases, resulting in significant overtreatment. 60-80% of elevated serum PSA findings are false-positives, as determined by prostate biopsy, thus demonstrating the inability of PSA alone to adequately discriminate between clinically significant PCa and benign diseases (Bastian et al 2009, Presti 2007). As a matter of fact, no single biomarker (PSA, its derivatives or other candidates) can fulfill the clinical needs of both high sensitivity and specificity currently. We have now found that combining another biomarker macrophage inhibitory cytokine 1 (MIC-1) with total serum PSA will improve the clinical specificity of PCa determination without compromising its high sensitivity.

MIC-1, also known as growth differentiation factor 15 (GDF15), is a protein belonging to the transforming growth factor beta superfamily (Bootcov et al. 1997) that has a role in regulating inflammatory and apoptotic pathways in injured tissues and during disease processes. MIC-1 is also known as TGF-PL, PDF, PLAB, and PTGFB. MIC-1 is over-expressed by many patients with common cancers including those of the prostate and can be further induced by cancer therapies including surgery, chemo and radio-therapy of prostate, colon and breast cancer (Bauskin et al. 2006, Breit et al. 2011). MIC-1 is linked to cancer in general and tumor expression of MIC-1 is often reflected in its blood levels, which increase with cancer development and progression (Welsh et al. 2003, Rasiah et al. 2006), generally in proportion to the stage and extent of disease. The role of MIC-1 in PCa is still unclear. Previous work has suggested that in established PCa, MIC-1mRNA expression is higher in Gleason sum >=7 tumors compared with lower-grade lesions (Nakamura et al. 2003). MIC-1 is highly expressed in human prostate cancer cell line LNPCa (Karan et al. 2003) and is found in high-grade prostatic intraepithelial neoplasia and in cancer cells but not in normal cells (Cheung et al. 2004). The possibility of using MIC-1 as a new biomarker for serum-based PCa test has been assessed (Brown et al 2006), although in contradiction with the current results MIC-1 serum level was found to be decreased in PCa patients in this study.

There is a continuing need in the art for high resolution method for detecting PCa. This need has been answered with the current invention.

### SUMMARY

Provided in an embodiment is a high resolution method of detecting prostate cancer comprising utilizing a solid phase immunoassay to determine if a patient fluid shows a MIC-1 value in Zone M (defined below). In an embodiment, the method comprises conducting a sandwich assay in an assay device that, if the determined value is in Zone M, automatically generates a report stating that a high risk of prostate cancer exists.

Also provided in an embodiment a high resolution method of detecting prostate cancer comprising utilizing a solid phase immunoassay to determine if a patient serum shows a MIC-1 value and a PSA value in Zone A (defined below) or, if utilized, Zone B (defined below). In an embodiment, the method comprises conducting a sandwich assay in an assay device that, if the determined value is in Zone A or, if utilized, Zone B, automatically generates a report stating that a high risk of prostate cancer exists.

In embodiments as to Zone M, Zone A or Zone B, the assay can be a sandwich assay, a particle-based sandwich assay, or an assay conducted utilizing as the solid phase a MTP, or a fluorescence-based assay, or an assay based on enzyme-generated signal.

In an embodiment, provided is a high resolution device for detecting prostate cancer comprising: (a) providing an electronic controller; (b) a data entry port for associating patient data with a solid phase immunoassay for patient fluid shows a MIC-1 levels; (c) an immunoassay detection device configured to read the result of the solid phase immunoassay; and (d) an output port configured for, if the controller determines that an immunoassay reading falls within Zone M, deliver a report stating that a high risk of prostate cancer exists.

In an embodiment, provided is a high resolution device for detecting prostate cancer comprising: (a) providing an electronic controller; (b) a data entry port for associating patient data with a solid phase immunoassay for MIC-1 and PSA levels; (c) an immunoassay detection device configured to read the result of the solid phase immunoassay; and (d) an output port configured for, if the controller determines that an immunoassay reading falls within Zone A or, if utilized, Zone B, deliver a report stating that a high risk of prostate cancer exists.

### DESCRIPTION OF THE DRAWINGS

The drawings illustrate important concepts of the invention, namely an approach to group assay data points into zones to facilitate a creation of a report that provides information about prostate cancer (FIGS. 1, 2 and 3) and the use of instrumentation to conduct the prostate cancer assay (FIG. 4). FIG. 5 provides an example of actual data and zones created using such instrumentation.

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIG. 1 depicts a version of 2-dimensional filter for identifying patients at especially high risk of prostate cancer (Zone A), at high but not especially high risk (Zone B*), and at low risk (Zone C*);

FIG. 2 depicts a version of 2-dimensional filter for identifying patients at especially high risk of prostate cancer (Zone A^{2*}), at high but not especially high risk (Zone B^{2*}), and at low risk (Zone C^{2*});

FIG. 3 depicts a version of 2-dimensional filter for identifying patients at especially high risk of prostate cancer (Zone A^{3*}), and at low risk (Zone C^{3*});

FIG. 4 schematically depicts a high resolution device for detecting prostate cancer;

FIG. 5 shows a data spread from subjects whose prostate was biopsied. Data corresponding to normal samples are shown with a ♦; data corresponding to patients whose biopsies were negative are shown with a ▴; data corresponding to patients whose biopsies showed a Gleason score of 6 is shown with a X; data corresponding to patients whose biopsies showed a Gleason score of 7 is shown with a [symbol] ; data corresponding to patients whose biopsies showed a Gleason score of 8 is shown with a ●; data corresponding to patients whose biopsies showed a Gleason score of 7 is shown with a ▪.

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation.

### DETAILED DESCRIPTION

While many protein cancer markers are known, choosing a proper one presents a challenge. The proper choice requires not only a useful biomarker, but also that the data interpretation facilitates clinical decisions. It is demonstrated below that MIC-1 is one such biomarker, and that a novel data analysis approach associated with it, and also with PSA, can aid patient care.

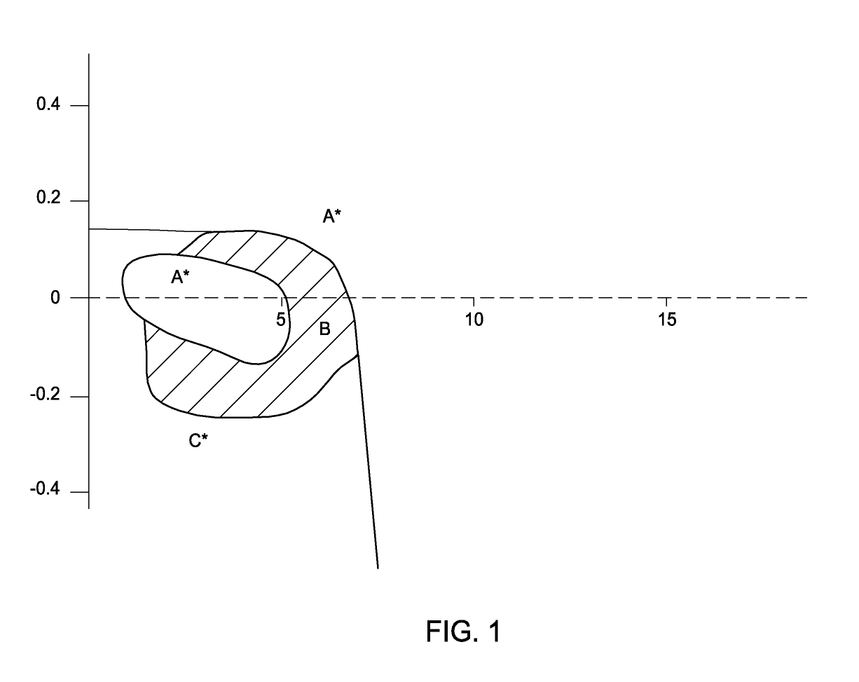

FIGS. 1, 2 and 3 depict values of the log₁₀ [MIC-1] in they axis, and values of [PSA] in the x axis. Concentrations ([ ]) are in ng/ml. The values are illustrated as those found in serum. Note that the y-axis, being in log₁₀ values, compacts the spread of the raw concentration values. The values can be as found in other bodily fluids, including tissue extracts.

Zone A can be made up of separate, non-overlapping zones, for example, zones A₁ and A₂. In embodiments, these zones defined as the regions where, for prostate biopsy tissue taken due to an increase in PSA concentration from one testing period to another, the values for the x and y axes corresponds to 80% or higher chance of the biopsy showing a Gleason value of 6 or higher. In embodiments, the value is a percentage of x % or higher, where x is a value from 80 to 90.

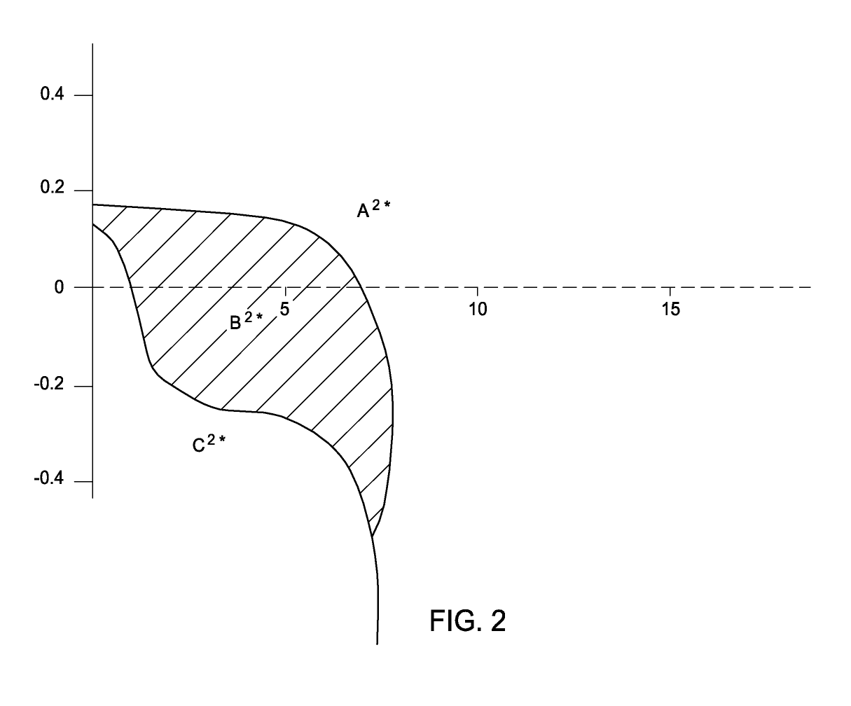

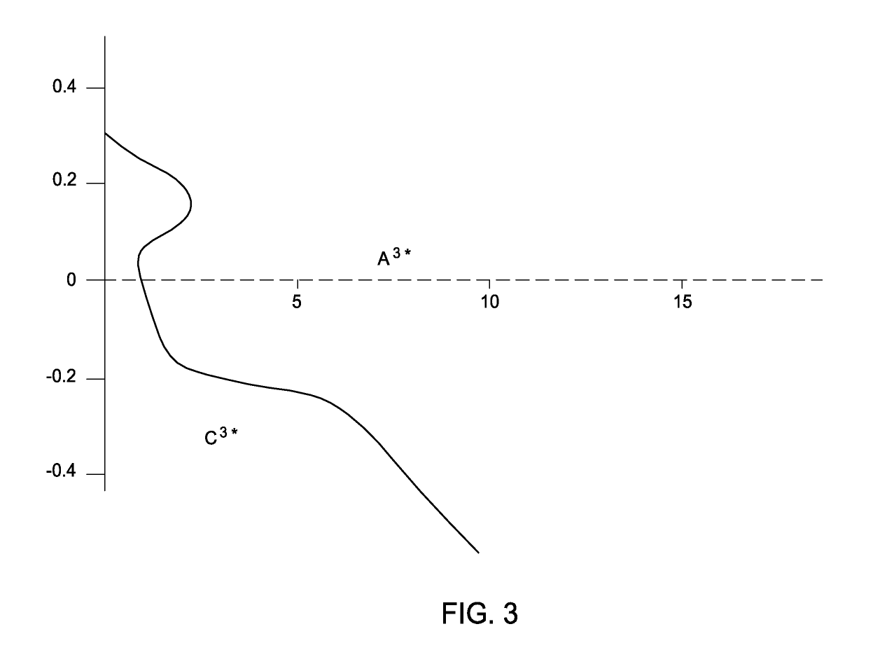

In embodiments, instead of Zone A, Zone A*, Zone A^{2*} or Zone A^{3*}, as illustrated in FIG. 1, FIG. 2 or FIG. 3, are utilized. In embodiments, Zone A³ is utilized. Zone A³ is comprised of Zone A^{∂}₁ and Zone A^{∂}₂. Zone A^{∂}₁ is the region where (a) log₁₀ [MIC-1] is a value ≧M*3, which can be illustrated as 0.173 and (b) Zone A^{∂}₂ is the region where [PSA] is ≧P₃, which can be illustrated as 7.33. FIGS. 1-3 are to scale.

Zone B is, in embodiments, the region less Zone A where, for prostate biopsy tissue taken due to an increase in PSA concentration from one testing period to another, the values for the x and y axes corresponds to 40% or higher chance of the biopsy showing a Gleason value of 6 or higher. In embodiments, the value is a percentage of x % or higher, where x is a value from 40 to 60.

In embodiments, instead of Zone B, Zone B* or Zone B^{2*}, as illustrated in FIG. 1 or FIG. 2, are utilized. In embodiments, Zone B^{∂} is utilized. Zone B^{∂} is a region bounded above and below by M₂ and M₁, respectively (here illustrated as 0.071 and −0.185, respectively), and right and left by P₁ and P₃, respectively (here illustrated as 1.26 and 7.33, respectively).

Zone C is, in embodiments, the region less Zones A and B where, for prostate biopsy tissue taken due to an increase in PSA concentration from one testing period to another, the values for the x and y axes corresponds to 20% or less chance of the biopsy showing a Gleason value of 6 or higher. In embodiments, the value is a percentage of x % or less, where x is a value from 5 to 20. In embodiments, instead of Zone C, Zone C*, Zone C^{2*} or Zone C^{3*}, as illustrated in FIG. 1, FIG. 2 or FIG. 3, are utilized.

Values for M₁, M₂, M₃, M₄, P₁, P₂ and P₃ can be, for example, a value in the following ranges (inclusive of the endpoints):

In embodiments, the low value to the high value range can be from any 0.01 increment within the range (including endpoints) to another such value in the range. As indicated, the boundaries M₁, M₂, etc. can be represented in a non-logarithmic scale. Similarly, the boundaries P₁, P₂, etc. could be represented in a logarithmic scale.

The Zones are established based on assay values associated with biopsy data, similar to the data reported herein. Those of skill will recognize that as further samples are assayed, the contours of the zones will become better focused, and may not have straight line boundaries. Thus, the refined zones are, for Zones A*, A** or A^{∂} or B*, B** or B^{∂}, substantially within the outer contours hereinabove defined (e.g., only ˜10% or less area of the log [MIC-1]×[PSA] area is outside the illustrated zone). For Zones corresponding to A₁ and A₂, we can arbitrarily set 0.6 as the upper boundary for measuring area, and 20 ng/mL as the right boundary for measuring area. With these designations, the refined Zone A*, A** or A^{∂} are substantially within the outer contours hereinabove defined (e.g., only ˜10% or less area of the log [MIC-1]×[PSA] area is outside the illustrated zone).

For other bodily fluids (such as without limitation urine, lymph, saliva, expectorate, tears, semen, intraocular fluid, tissue extracts, and the like), the boundaries of Zones A, B and C are separately determined.

In embodiments, the PSA measured is total PSA.

In embodiments focusing on MIC-1 without reference to PSA, Zone M is the zone in which, for prostate biopsy tissue taken due to an increase in PSA concentration from one testing period to another, the values for the y axe corresponds to 40% or higher chance of the biopsy showing a Gleason value of 6 or higher. In embodiments, the value is a percentage of x % or higher, where x is a value from 40 to 90. In embodiments, Zone M* is used in place of Zone M, where Zone M* is where log [MIC-1] is ≧M₄, or M₃, or M₂.

In embodiments, the measurements of the invention are obtained with a solid phase immunoassay. By “solid phase immunoassay” it is meant that the assay depends on one of an antibody and its cognate antigen being bound, adsorbed, linked to or otherwise stably associated with a solid phase.

In embodiments, the measurements of the invention are made with a sandwich assay. By “sandwich” assay it is meant that one binding entity (a high specificity binding moiety, typically an antibody, or a derivative expressed from an antibody gene or its segment, or a DNA fragment having sequence homology to the antibody gene), binds one portion of the analyte, and a separate binding entity binds to another portion of the analyte. Detection is dependent on formation of the sandwich, the top layer of which often includes a label (color dye, fluorescent dye, fluorescent protein, fluorescent nanostructure, etc.).

In a sandwich assay detection of the sandwich can be dependent on the proximity of the second binding entity to the first binding entity. In some cases, proximity is established because (a) the first binding entity is attached or bound to a solid support such that the particular solid support or the region of the solid support identifies what binding entity is there, and (b) the second binding entity has a detectable moiety whose detection at the support or region establishes proximity. In some cases, for example, both binding entities have moieties that interact with proximity to establish a signal. For example, one binding moiety can have a donor moiety and the other an acceptor moiety for generating a FRET signal

A “particle-based” sandwich assay is one utilizing suspendible particles to which first binding moieties are attached or bound, where the particles can be identified for their corresponding first binding moieties by color, shape, bar code, 2D bar codes, other multi-dimensional bar codes, electronic circuitry in the particles, or the like.

Such particle-based assays can include assays utilizing the light-triggered microtransponders (“MTPs”) and flow reading apparatus described in Lin et al., Clinical Chemistry 2007, v. 53, p. 1372-1376. Or, such particle-based assays can include assays utilizing the MTPs in the compact analyzer described in U.S. Ser. No. 61/713,825, filed 15 Oct. 2012. One brand of MTP is the p-Chip® transponder available from PharmaSeq, Inc., Monmouth Jct., N.J.

In embodiments, the assays of the invention are conducted utilizing silver nanoparticle-enhanced fluorescence, such as outlined in Li et al., Anal. Bioanal. Chem. 2010, v. 398, p. 1993-2001 and Mandecki et al., U.S. Pat. Publ. US 2013-012311. Fluorescence emission can be dramatically altered/enhanced by the oscillating charge in a nearby metallic particle. This magnifying effect can be explained theoretically by considering the change of the photonic mode density near the fluorophore due to coupling to the conducting surface. Total effects include increased rates of excitation, increased quantum yields, and decreased fluorescence lifetimes, all of which lead to high fluorescence signal enhancement and significantly decreased photobleaching. PharmaSeq's results show that the net gain in fluorescence signal in some cases can be over 100-fold. See Li et al. It is expected that using localized enhanced excitation in proximity to plasmonic platforms will dramatically increase the signal, thereby providing excellent sensitivity of fluorescence detection from MTPs.

The report stating that a high risk of prostate cancer exists can take a number of forms. It can be a statement that a prostate biopsy for the patient is recommended. The report may simply recite for example “Biopsy Needed”, or “Biopsy Recommended”, or “Refer to Dr. @” (where Dr. @ is a urologist). The context of PSA testing will establish that these statements are in reference to a prostate cancer risk.

### High Resolution Detection Device

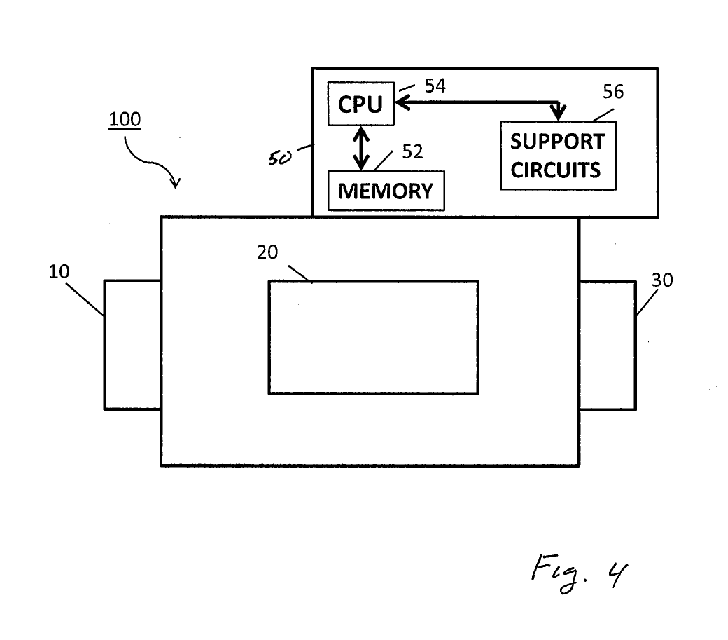

For detecting cancer risk associated with MIC-1, or the combination of MIC-1 and PSA, a detection device that integrates a report on cancer risk can be used. For example, the device 100 can have a data entry port 10 (FIG. 4) through which the device, or its associated electronics, receives patient data, such as, one or more of name, age, weight, prostate hyperplasia, medical conditions, and the like. The entry port can be by way of an electronic network, wherein the data is inputed or taken from a database at a workstation or other electronic device and directed to the immunoassay detection device or marked for association with the immunoassay to be conducted on the immunoassay detection device.

An immunoassay detection port 20 can comprise the systems that detect the result of the assay, such as one or more light sources to direct light to assay surfaces or assay vessels for obtaining reflectance, optical density or fluorescence data indicative of MIC-1 or PSA amount. With optical detection, the device will typically include one or more light detection devices. The immunoassay detection port can include more features that support the assay reactions, such as temperature control, mixing, and the like.

The output port 30 can be an output screen, a printer, or a communication link (which can share communication pathways with the data entry port). As a communication link, it can for example direct a report formulated by the controller 50 to a database that associates the results with the patient, or can direct a communication such as an email to the patient or his or her care provider.

The device 100 has controller 50 (FIG. 4), which can comprise a central processing unit (CPU) 54, a memory 52, and support circuits 56 for the CPU 54 and is coupled to and controls the device 100 or, alternatively, operates to do so in conjunction with computers (or controllers) connected to the device 100. For example, another electronic device can supply software, or operations may be calculated off-site with controller 50 coordinating off-sight operations with the local environment. The controller 50 may be one of any form of general-purpose computer processor, or an array of processors, that can be used for controlling various devices and sub-processors. The memory, or computer-readable medium, 52 of the CPU 54 may be one or more of readily available memory such as random access memory (RAM), read only memory (ROM), flash memory, floppy disk, hard disk, or any other form of digital storage, local or remote. The support circuits 56 are coupled to the CPU 54 for supporting the processor in a conventional manner. These circuits can include cache, power supplies, clock circuits, input/output circuitry and subsystems, and the like. Methods of operating the analyzer may be stored in the memory 52 as software routine that may be executed or invoked to control the operation of the immunization testing device 100. The software routine may also be stored and/or executed by a second CPU (not shown) that is remotely located from the hardware being controlled by the CPU 54. While the above discussion may speak of the “controller” taking certain actions, it will be recognized that it may take such action in conjunction with connected devices (e.g., the controller physically on the device 100 may have limited capacity, and serve mostly to coordinate communication with more powerful processor(s)).

All ranges recited herein include ranges therebetween, and can be inclusive or exclusive of the endpoints. Optional included ranges are from integer values there between (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more.

Specific embodiments according to the methods of the present invention will now be described in the following examples. The examples are illustrative only, and are not intended to limit the remainder of the disclosure in any way.

### Example 1—MIC-1 Assay

### Antibodies and Antigen

Anti-MIC-1 (MAB957, R&D Systems) was used as the capture antibody and conjugated to the polymer coated p-Chip microtransponders (MTPs, see Lin et al., Clinical Chemistry 2007, v. 53, p. 1372-1376). Recombinant MIC-1 protein from R&D Systems (957-GD) was used as the antigen and spiked in 1:4 diluted pooled normal human male serum (Bioreclamation) for building the standard curve. Biotinylated anti-MIC-1 (BAF940, R&D Systems) was used as the detection antibody and subsequently stained by streptavidin-phycoerythrin (SAPE) (Invitrogen).

### Serum Samples

A total of 70 serum samples were acquired from the JHU Brady Urologic Institute biorepository that consisted of 5 groups with 14 cases per group of normal, biopsy negative, PCa patients with PSA<2.5 ng/ml, PSA 2.5-10 ng/ml and PSA>10 ng/ml. Out of the 42 PCa patients, 19 of them Gleason scored 6, 14 have Gleason score 7, 5 have Gleason score 8 and 4 have Gleason score 9. The serum samples were diluted to 1:4 in the test to minimize serum interference and the test results of MIC-1 level are summarized in Table 1. The associated PSA levels and Gleason scores were retrieved from the database of the JHU Brady Urologic Institute biorepository.

p-Chip MTPs and Simuplex

In this particular implementation, the assay was conducted using the p-Chip MTPs and Simuplex analyzer available from PharmaSeq, Inc., (Monmouth Junction, N.J.).

Simuplex analyzer is a unique particle-based, multiplex platform that can be used for the analysis of various bio-molecules (nucleic acids, proteins, small chemical molecules). The system is based on small electronic devices, p-Chip® MTPs, along with a unique fluorescence and radio frequency (RF) readout (flow reader) for the p-Chip® MTPs. The p-Chip® MTP is a silicon-based monolithic, light-activated 500×500×100 μm integrated circuit that can transmit its identification code at a fixed radio frequency. Each chip consists of photocells, read-only memory (ROM) that contains the ID, logic circuitry and an integrated antenna. Visible light, typically from a red or green laser source, is pulsed over the range 0.5-5.5 MHz to provide power and a stable clocking signal for the logic circuitry. The photocells, when illuminated, provide power for electronic circuits on the chip that modulate the current through the antenna in a way that is dependent on the ROM contents. The antenna transmits the ID through a varying magnetic field induced as a result of the modulated current in the antenna. The resulting variable magnetic field in the vicinity of the p-Chip can then be measured with a nearby coil/receiver device and decoded using specialized firmware and software to provide the ID value, which in turn identifies the analyte immobilized on the p-Chip using an assay-specific database. The use of p-Chip® MTPs to analyze biological samples is described in more detail in four recent papers [Lin X et al., 2007. Clin Chem 53:1372-1376; Li J et al., 2010. Anal Bioanal Chem. 398(5):1993-2001; Rich R et al., 2012. Anal Bioanal Chem, 404(8):2223-2231; Mandecki W et. al., 2006. Cytometry Part A, 69A:1097-1105]. p-Chip® MTPs have been also used for tagging of small objects and laboratory animals [Jolley-Rogers G, et al., 2012. Zootaxa, 3359:31-42; Gruda M C et al., 2010. J Am Assoc Lab Anim Sci, 49:826-831; Robinson E J H et al., 2009. Behav Ecol Sociobiol, 63(5) 627-636].

A suspension of p-Chip® MTPs is analyzed by repeatedly passing it through a narrow channel on an analyzer, named “flow reader” or “Simuplex™”, which both reads the ID values and collect fluorescence measurements. The flow reader was designed to support transfer rates of up to 1,000 p-Chip® MTPs/sec. Although the times needed to read an ID and measure fluorescence can be as short as 500 ps and 1-2 ms, respectively, the actual sustained readout times for both fluorescence and ID are extended to allow for prolonged sampling. Processing a single sample with up to several hundred p-Chip® MTPs takes five minutes or less. The current instrument configured for two-color detection, i.e., 532 nm and 635 nm Cy3/Cy5.

Polymer Coating and Amino Group Conversion on the p-Chip® MTPs

p-Chip® MTPs were pretreated with 99.5% methyl alcohol at room temperature (RT) for 10 min, and repeated three times. The p-Chip® MTPs were then rinsed with 0.01% distilled water and 0.9% aminopropyltriethoxysilane (APTS) in dry toluene/dimethylformamide (DMF) mixture at RT, and repeated four times. After rinsing, p-Chip® MTPs were immediately treated with a coating solution (mixture of 0.01% distilled water, 0.9% APTS, and 0.3% 3-glycidoxypropyltrimethoxysilane (GPTS) in dry toluene and DMF) at 80° C. for 45 min and repeated once. After the coating reaction, p-Chip® MTPs were washed once with toluene, three times with DMF, and three times with acetonitrile at RT, followed by air drying. The procedure placed both amino and hydroxy groups on the surface of p-Chip® MTPs. Amino-derivatized p-Chip® MTPs were treated with 10% succinic anhydride in dry pyridine:DMF (1:9) on a tissue culture rotator at RT for 30 min. This step was repeated once using fresh reagents. After the reaction, the carboxylated p-Chip® MTPs (amines converted to carboxylic acids) were washed with DMF four times and acetonitrile twice, followed by air drying.

### Assay Procedure

The assay is a sandwich solid phase immunoassay implemented on p-Chip® MTPs as solid phase. p-Chip® MTPs carry a unique ID in their electronic memory, and the ID is capable of identifying the solid phase particle and also biochemical processes occurring on the solid phase. The p-Chip® MTPs are first conjugated to a capture antibody. In the assay, such derivatized p-Chip® MTPs are incubated with a sample containing a biomarker and the biomarker is captured by the capture antibody. In the next two assay steps, a detection antibody conjugated to biotin is added, followed by a staining reagent, streptavidin conjugated to phycoerythrin. Thus, the sandwich formed includes the p-Chip® MTP solid phase, capture antibody, biomarker, detection antibody and staining reagent. Then, in the fluorescence quantification step, the p-Chip® MTPs are run in a PharmaSeq flow reader Simuplex, and resulting data on the biomarker concentration are presented in a tabulated form.

Anti-MIC-1 capture antibody was conjugated to polymer coated p-Chip® MTPs and incubated with 40 ul 1:4 diluted serum sample for 1 hr. To build the standard curve, recombinant MIC-1 antigen with a series of dilutions was spiked in 1:4 diluted pooled normal human male serum. The chips were washed with TBS-Tween solution (“TBST”) for 3 times after the sample incubation and then incubate with biotinylated anti-MIC-1 detection antibody for 1 hr. In the next step the chips were subjected to TBST washing for 3 times and stained with streptavidin-phycoerythrin (SA-PE) for 30 min. All the chips were pooled together in the end of the assay, washed 2 times by TBST and subjected to PharmaSeq flow reader Simuplex™ for signal detection and analysis.

### Results

All 70 serum samples provided by JHU Brady Urologic Institute biorepository were tested by MIC-1 assay described above. The MIC-1 levels were summarized in Table 1. The associated PSA levels and Gleason scores were retrieved from the database of the JHU Brady Urologic Institute biorepository.

TABLE 1
Levels of MIC-1 and PSA in 70 serum samples. Concentration unit:
ng/ml. BX-ve: biopsy negative.

| | | Sample | | | |
|---|---|---|---|---|---|
| | | ID | PSA | Gleason | MIC-1 |
| Normal | 1 | 8430 | 2.3 | | 0.668 |
| | 2 | 7033 | 1.8 | | 1.248 |
| | 3 | 6990 | 2.3 | | 1.188 |
| | 4 | 5812 | 0.74 | | 0.632 |
| | 5 | 5810 | 0.86 | | 0.908 |
| | 6 | 5807 | 0.42 | | 0.644 |
| | 7 | 5805 | 0.49 | | 0.884 |
| | 8 | 5804 | 0.32 | | 1.200 |
| | 9 | 6844 | 1.41 | | 1.004 |
| | 10 | 6847 | 1.03 | | 0.702 |
| | 11 | 6848 | 0.25 | | 0.534 |
| | 12 | 6851 | 0.33 | | 1.332 |
| | 13 | 6886 | 1.48 | | 1.315 |
| | 14 | 6892 | 0.64 | | 0.692 |
| | Mean | | 1.026 | | 0.925 |
| | STDEV | | 0.718 | | 0.286 |
| | Mean | | 0.506 | | 0.853 |
| | (PSA < 1) | | | | |
| | STDEV | | 0.219 | | 0.286 |
| | (PSA < 1) | | | | |
| BX-ve | 1 | 9270 | 6.1 | | 0.740 |
| | 2 | 9226 | 5.5 | | 1.156 |
| | 3 | 9217 | 3.9 | | 1.056 |
| | 4 | 9216 | 6.44 | | 0.840 |
| | 5 | 8985 | 4.4 | | 1.336 |
| | 6 | 8971 | 10.5 | | 1.448 |
| | 7 | 8928 | 19.4 | | 1.248 |
| | 8 | 8885 | 4.8 | | 0.436 |
| | 9 | 11122 | 4.1 | | 0.692 |
| | 10 | 11129 | 7.1 | | 0.335 |
| | 11 | 11240 | 4.8 | | 0.611 |
| | 12 | 11241 | 4.3 | | 0.642 |
| | 13 | 11488 | 4.7 | | 0.930 |
| | 14 | 11489 | 3.8 | | 0.898 |
| | Mean | | 6.417 | | 0.883 |
| | STDEV | | 4.132 | | 0.335 |
| PSA < 2.5 | 1 | 8721 | 1.39 | 6 | 1.152 |
| | 2 | 8665 | 1.89 | 6 | 0.748 |
| | 3 | 8623 | 2.46 | 6 | 2.068 |
| | 4 | 7687 | 1.4 | 6 | 1.016 |
| | 5 | 7101 | 0.1 | 6 | 1.396 |
| | 6 | 7038 | 2.3 | 6 | 1.056 |
| | 7 | 6610 | 1.9 | 8 | 0.992 |
| | 8 | 6202 | 1.7 | 6 | 1.868 |
| | 9 | 11120 | 1.4 | 9 | 1.061 |
| | 10 | 11124 | 2.1 | 9 | 2.159 |
| | 11 | 11131 | 1.7 | 6 | 1.061 |
| | 12 | 11466 | 2.17 | 6 | 0.656 |
| | 13 | 11505 | 2.3 | 6 | 1.016 |
| | 14 | 11511 | 2.35 | 9 | 1.148 |
| | Mean | | 1.797 | | 1.243 |
| | STDEV | | 0.613 | | 0.465 |
| PSA = 2.5-10 | 1 | 9295 | 5.5 | 6 | 1.524 |
| | 2 | 9288 | 3.7 | 6 | 1.776 |
| | 3 | 9287 | 4.6 | 7 | 0.752 |
| | 4 | 9286 | 5 | 7 | 0.832 |
| | 5 | 9285 | 2.6 | 7 | 1.764 |
| | 6 | 9246 | 4 | 7 | 1.924 |
| | 7 | 9195 | 5.3 | 6 | 2.756 |
| | 8 | 9194 | 3.3 | 6 | 1.132 |
| | 9 | 11467 | 9.1 | 7 | 2.070 |
| | 10 | 11468 | 4.6 | 6 | 0.685 |
| | 11 | 11469 | 8.6 | 7 | 0.628 |
| | 12 | 11472 | 3.9 | 7 | 1.095 |
| | 13 | 11473 | 4.7 | 7 | 0.922 |
| | 14 | 11487 | 6.6 | 7 | 1.013 |
| | Mean | | 5.107 | | 1.348 |
| | STDEV | | 1.870 | | 0.633 |
| PSA > 10 | 1 | 8666 | 12 | 7 | 1.056 |
| | 2 | 8646 | 17 | 7 | 1.552 |
| | 3 | 8641 | 241.3 | 9 | 2.460 |
| | 4 | 6008 | 12.1 | 6 | 1.576 |
| | 5 | 6003 | 10 | 6 | 2.600 |
| | 6 | 5941 | 13.6 | 8 | 1.436 |
| | 7 | 5744 | 11.9 | 6 | 1.904 |
| | 8 | 5696 | 11.9 | 8 | 2.700 |
| | 9 | 11355 | 13.8 | 7 | 1.579 |
| | 10 | 11403 | 10.9 | 8 | 1.121 |
| | 11 | 11426 | 17.9 | 7 | 1.838 |
| | 12 | 11443 | 19.6 | 6 | 1.912 |
| | 13 | 11540 | 13.9 | 8 | 1.330 |
| | 14 | 11619 | 12.2 | 7 | 1.030 |
| | Mean | | 29.864 | | 1.721 |
| | STDEV | | 60.918 | | 0.551 |
| PCa | Mean | | 8.842 | | 1.224 |
| patients all | STDEV | | 28.625 | | 0.552 |
| Gleason 6 | Mean | | 4.916316 | 6 | 1.468551 |
| Gleason 7 | Mean | | 8.714286 | 7 | 1.289614 |
| Gleason 8 | Mean | | 10.44 | 8 | 1.515763 |
| Gleason 9 | Mean | | 61.788 | 9 | 1.706847 |

To compare the results, the prostate cancer patient samples were displayed in three categories with different PSA levels (PSA<2.5 ng/ml, PSA=2.5-10 ng/ml and PSA>10 ng/ml) as well as one pooled group. MIC-1 exhibits higher protein levels in pooled patient group than normal and biopsy negative group (average 0.925 ng/ml in normal group, average 0.883 ng/ml in biopsy negative group and average 1.224 ng/ml in pooled PCa patients). In addition, MIC-1 levels correlate with PSA levels very well (average 1.243 ng/ml in PCa patient with PSA<2.5 ng/ml, average 1.348 ng/ml in PCa patient with PSA 2.5-10 ng/ml and average 1.721 ng/ml in PCa patient with PSA>10 ng/ml).

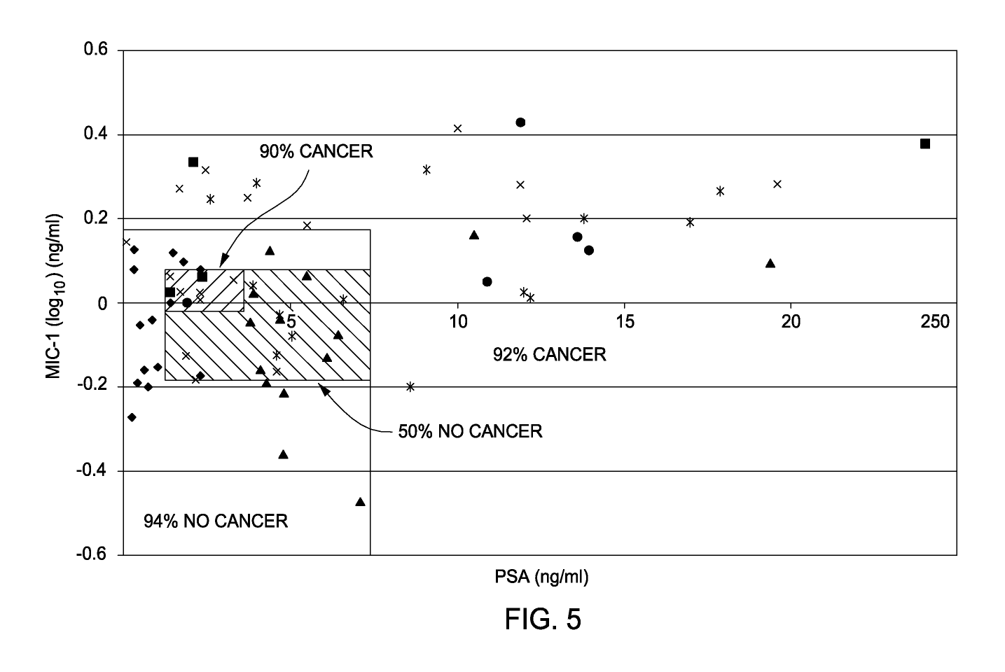

The logarithmic value of MIC-1 level was plotted against PSA level for each sample in a 2D plot and several zones were identified in the 2D plot for PCa patients (FIG. 5). Different zones indicate the likelihood that the patient has prostate cancer.

### Example 2—Colorimetric MIC-1 Assay in Microtiter Plate

### Antibodies, Antigen and Serum Samples

These components are as described in Example 1.

### Procedures

Wells in the microtiter plate are coated with 50 ul of anti-MIC-1 capture antibody (1-4 μg/ml) at 4° C. overnight followed by washing and blocking. Then 50 ul 1:4 diluted serum samples or protein standards (prepared by spiking recombinant MIC-1 in pooled normal human male serum diluted 1:4). The plates are then sealed with plastic plate sealer and incubated at 37° C. for 90 minutes followed by PBS-Tween 20 (0.05%) washing for 5 times. 50 μl of biotinylated anti-MIC-1 detection antibody (0.2-0.6 μg/ml) were then added and incubated at room temperature for 30-40 minutes. The plates are then washed again with PBS-Tween 20 (0.05%) for 5 times and incubated with 50 μl of streptavidin-HRP (1:200 dilution) at room temperature for 30 minutes. In the end the plates are washed with PBS-Tween 20 (0.05%) for 5 times and incubated with 50 μl of TMB substrate in dark with frequent checking. When the adequate color develops, the reaction is stopped with 25 μl of TMB “stop solution” and the plates are read at 450 nM in an automated ELISA microplate reader.

### Example 3—Analysis of Data

A product of a diagnostic assay that measures concentrations of MIC-1 and PSA in several serum samples is a group of pairs of numbers (xᵢ, yᵢ), i=1, . . . , N, where xᵢ and yᵢ are the two biomarker concentrations, and N is the number of samples. The data can be plotted on a 2-dimensional graph where the axes are the concentrations [or the logarithm(s) thereof]. The assay was performed on 70 retrospective samples obtained from Johns Hopkins University, and the data were plotted and separated into different prostate cancer (PCa) risk groups, as illustrated in FIGS. 1-3.

Results of the analysis where the grouping was done with respect to the MIC-1 concentration is presented in FIG. 5. The samples were assigned to three groups, cancer (100% true positives rate in this group), no cancer (70% true negatives rate) and an intermediate (non-conclusive) group (white area in FIG. 1 and FIG. 2).

Similarly, FIG. 5 presents the analysis results for the grouping done with respect to the PSA concentration. The first major conclusion from a comparison is that MIC-1 is a better predictor of PCa than PSA, for the 70 samples obtained from JHU. Indeed, the percentages of true positives and true negatives are higher for MIC-1 than PSA: 100% and 70% versus 89% and 50%, respectively, and the number of samples not assigned to a category is similar, 29 and 24, respectively.

An inspection of data in FIG. 5 indicates the presence of a cancer “hot spot” at medium MIC-1 and low-medium PSA concentrations. This hot spot is surrounded by an area comprising mostly no-cancer points (green zone in FIG. 5) or a mixed area (blue zone; non-conclusive). Clear limits for the zones are marked.

Based on such defined three zones, key characteristics of the cancer determination are given in Table 2.

TABLE 2
Characteristics of cancer determinations. The zones
are defined in FIG. 5.

| | | | | |
|---|---|---|---|---|
| A | Total number of samples | 100% | (N = 70) | |
| B | True positives (cancer) | 92% | (33/36) | |
| C | False positives | 8% | (3/36) | (biopsies were not |
| | | | | needed for patients |
| | | | | in this category) |
| D | True negatives | 94% | (17/18) | |
| E | False negatives | 6% | (1/18) | (patients with cancer |
| | | | | not properly identified |
| | | | | as such) |
| F | Not assigned | 23% | (16) | |

### Example 4—Gleason Score Analysis

The data of MIC-1 level and PSA level was plotted on a 2-dimensional graph as described in Example 3. Further inspection of data in FIG. 5 suggests that cases of Gleason score 6 and 7 are well separated in two zones where 85% cases of Gleason score 6 reside in white zone and 85% cases of Gleason score 7 are in red zone. The zones (two or more) for Gleason scores can be valuable for prognosis of PCa.

### Further Embodiments

The invention is further described with reference to a number of embodiments of the following letter identifiers:

### Embodiment A

A high resolution method of detecting prostate cancer comprising utilizing a solid phase immunoassay to determine if a patient fluid shows a MIC-1 value in Zone M. For example, assaying can be by solid phase assay, or by sandwich assay, or by another assay method. In embodiments, Zone M is substituted with Zone M*.

### Embodiment B

A high resolution method of detecting prostate cancer comprising utilizing a solid phase immunoassay to determine if a patient serum shows a MIC-1 value and a PSA value in Zone A or, if utilized, Zone B. For example, assaying can be by solid phase assay, or by sandwich assay, or by another assay method. In embodiments, there is no Zone B. In embodiments, there is a Zone A and Zone B. In embodiments, Zone A is substituted with Zone A*, A** or A⁷. In embodiments, Zone A is substituted with Zone B*, B** or B^{∂},

### Embodiment C

The high resolution method of detecting prostate cancer of Embodiment A or B, wherein the immunoassay is a sandwich assay.

### Embodiment D

The high resolution method of detecting prostate cancer of Embodiment A, B or C, wherein the solid phase assay is particle-based assay.

### Embodiment E

The high resolution method of detecting prostate cancer of Embodiment A-C or D, wherein the assay is based on enzyme-generated signal.

### Embodiment F

The high resolution method of detecting prostate cancer of Embodiment A-D or E, wherein the assay is fluorescence-based.

### Embodiment G

The high resolution method of detecting prostate cancer of Embodiment A-E or F, wherein the assay is conducted on serum.

### Embodiment H

The high resolution method of detecting prostate cancer of Embodiment A, further comprising conducting the sandwich assay in an assay device that, if the determined value is in Zone M, automatically generates a report stating that a high risk of detecting prostate cancer exists.

### Embodiment I

The high resolution method of detecting prostate cancer of Embodiment B, further comprising conducting the sandwich assay in an assay device that, if the determined value is in Zone A or, if utilized, Zone B, automatically generates a report stating that a high risk of detecting prostate cancer exists.

### Embodiment J

The high resolution method of detecting prostate cancer of Embodiment H or I, wherein the immunoassay is a sandwich assay.

### Embodiment K

The high resolution method of detecting prostate cancer of Embodiment H, I or J, wherein the solid phase assay is particle-based assay.

### Embodiment L

The high resolution method of detecting prostate cancer of Embodiment H-J or K, wherein the assay is based on enzyme-generated signal.

### Embodiment M

The high resolution method of detecting prostate cancer of Embodiment H-K or L, wherein the assay is fluorescence-based.

### Embodiment N

The high resolution method of detecting prostate cancer of Embodiment H-L or M, wherein the assay is conducted on serum.

### Embodiment O

A high resolution device for detecting prostate cancer comprising:

providing an electronic controller;

a data entry port for associating patient data with a solid phase immunoassay for patient fluid shows a MIC-1 levels;

an immunoassay detection device configured to read the result of the solid phase immunoassay; and

an output port configured for, if the controller determines that an immunoassay reading falls within Zone M, deliver a report stating that a high risk of detecting prostate cancer exists.

### Embodiment P

A high resolution device for detecting prostate cancer comprising:

providing an electronic controller;

a data entry port for associating patient data with a solid phase immunoassay for MIC-1 and PSA levels;

an immunoassay detection device configured to read the result of the solid phase immunoassay; and

an output port configured for, if the controller determines that an immunoassay reading falls within Zone A or, if utilized, Zone B, deliver a report stating that a high risk of detecting prostate cancer exists.

### Embodiment Q

The high resolution device of Embodiment O or P, wherein the immunoassay of the detection device is a sandwich assay.

### Embodiment R

The high resolution device of Embodiment O, P or Q, wherein the solid phase assay of the detection device is particle-based assay.

### Embodiment S

The high resolution device of Embodiment O-Q or R, wherein the assay of the detection device is based on enzyme-generated signal.

### Embodiment T

The high resolution device of Embodiment O-R or S, wherein the assay of the detection device is fluorescence-based.

### Embodiment U

The high resolution device of Embodiment O-S or T, wherein the assay of the detection device is conducted on serum.

This invention described herein is of a high resolution prostate cancer assay method. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

### REFERENCES

American C (2011) Cancer Facts & Figures. Atlanta: American Cancer Society.
Bootcov M R, Bauskin A R, Valenzuela S M, Moore A G, Bansal M, et al. (1997) MIC-1, a novel macrophage inhibitory cytokine, is a divergent member of the TGF-beta superfamily. Proc Natl Acad Sci USA 94: 11514-11519.
Bauskin A R, Brown D A, Kuffner T, Johnen H, Luo X W, et al. (2006) Role of macrophage inhibitory cytokine-1 in tumorigenesis and diagnosis of cancer. Cancer Res 66: 4983-4986.
Breit S N, Johnen H, Cook A D, Tsai V W, Mohammad M G, et al. (2011) The TGF-beta superfamily cytokine, MIC-1/GDF15: a pleotrophic cytokine with roles in inflammation, cancer and metabolism. Growth Factors 29: 187-195.
Welsh J B, Sapinoso L M, Kern S G, Brown D A, Liu T, et al. (2003) Large-scale delineation of secreted protein biomarkers overexpressed in cancer tissue and serum. Proc Natl Acad Sci USA 100: 3410-3415.
Rasiah K K, Kench J G, Gardiner-Garden M, Biankin A V, Golovsky D, et al. (2006) Aberrant neuropeptide Y and macrophage inhibitory cytokine-1 expression are early events in prostate cancer development and are associated with poor prognosis. Cancer Epidemiol Biomarkers Prey 15: 711-716.
Nakamura T, Scorilas A, Stephan C, et al. Quantitative analysis of macrophage inhibitory cytokine-1 (MIC-1) gene expression in human prostatic tissues. BrJCancer 2003; 88:1101-4.
Karan D, Chen S J, Johansson S L, et al. Dysregulated expression of MIC-1/PDF in human prostate tumor cells. Biochem Biophys Res Commun 2003; 305:598-604.
Cheung P K, Woolcock B, Adomat H, et al. Protein profiling of microdissected prostate tissue links growth differentiation factor 15 to prostate carcinogenesis. Cancer Res 2004; 64:5929-33.
Brown D A, Stephan C, Ward R L, et al. Measurement of serum levels of macrophage inhibitory cytokine 1 combined with prostate-specific antigen improves prostate cancer diagnosis. Clin Cancer Res 2006; 12:89-96.
Jemal A, Siegel R, Hao Y, Xu J, and Thun M J. 2010. Cancer statistics, C A Cancer J Clin 2010; 60:277-300.
Etzioni R, Penson D F, Legler J M, et al. 2002. Overdiagnosis due to prostate specific antigen screening: lessons from U.S. prostate cancer incidence trends. J Natl Cancer Inst 94:981-90.
Bastian P J, Carter B H, Bjartell A, et al. 2009. Insignificant prostate cancer and active surveillance: from definition to clinical implications. Eur Urol. 55:1321-1332.
Klotz L. 2010 Active surveillance for prostate cancer: A review. Curr Urol Rep 11:165-17.
Presti J C Jr. 2007. Prostate biopsy: current status and limitations. Rev. Urol. 9(3): 93-98.
Lin X, Flint J, Azaro M, Coradetti T, Kopacka W, Streck D, Wang Z, Dermody J, and Mandecki W. 2007. Microtransponder-based multiplex assay for genotyping cystic fibrosis. Clin Chem 53:1372-1376.
Li J, Wang Z, Gryczynski I, Mandecki W. 2010. Silver nanoparticle-enhanced fluorescence in microtransponder-based immuno- and DNA hybridization assays. Anal Bioanal Chem. 398(5):1993-2001.
Rich R, Li J, Fudala R, Gryczynski Z, Gryczynski I, Mandecki W (2012) Properties of coatings on RFID p-Chips that support plasmonic fluorescence enhancement in bioassays. Anal Bioanal Chem 404(8):2223-2231; PMID: 22960796.
Mandecki W, Ardelt B, Coradetti T, Davidowitz H, Flint J, Huang Z, Kopacka W, Lin X, Wang Z, and Darzynkiewicz Z (2006) Microtransponders, the miniature RFID electronic chips, as platforms for cell growth in cytotoxicity assays. Cytometry Part A 69A:1097-1105.
Jolley-Rogers G, Yeates D K, Crost J, Cawsey E M, Suter P, Webb J, Morris R G, Qian Z, Rodriguez E and Mandecki W (2012) Ultra-small RFID p-Chips on the heads of entomological pins provide an automatic and durable means to track and label insect specimens. Zootaxa 3359:31-42.
Gruda M C, Pinto A, Craelius A, Davidowitz H, Kopacka W M, Li J, Qian J, Rodriguez E, Kuspiel E and Mandecki W (2010) A system for tagging laboratory mice with light-activated microtransponders. J Am Assoc Lab Anim Sci 49:826-831.
Robinson E J H. Thomas O. Richardson T O, Sendova-Franks A B, Feinerman O, and Franks N R. 2009. Radio tagging reveals the roles of corpulence, experience and social information in ant decision making. Behav Ecol Sociobiol, 63(5) 627-636.
Robinson E H, Mandecki W (2011) Distributed decisions: New insights from radio-tagged ants. In: Ant Colonies: Behavior in Insects and Computer Applications. Ed. E C Sun, pp. 109-128, Nova Science Publishers, Hauppauge, N.Y.

1. A high resolution method of detecting prostate cancer comprising conducting a particle-based, solid phase sandwich immunoassay for MIC-1 in patient serum; and determining if a patient serum shows a MIC-1 value in Zone M.

2. The high resolution method of prostate cancer of claim 1, wherein the immunoassay is fluorescence-based.

3. (canceled)

4. The high resolution method of prostate cancer of claim 2, further comprising conducting the sandwich assay in an assay device that, if the determined value is in Zone M, automatically generates a report stating that a high risk of prostate cancer exists.

5. (canceled)

6. A high resolution method of detecting prostate cancer comprising conducting a particle-based solid phase sandwich immunoassay for MIC-1 and PSA in patient serum; and determining if a patient serum shows a MIC-1 value and a PSA value in Zone A.

7-8. (canceled)

9. The high resolution method of prostate cancer of claim 8, further comprising conducting the sandwich assay in an assay device that, if the determined value is in Zone A or, if utilized, Zone B, automatically generates a report stating that a high risk of prostate cancer exists.

10. The high resolution method of prostate cancer of claim 8, wherein the sandwich assay is particle-based assay.

11. The high resolution method of prostate cancer of claim 10, wherein the assay is conducted utilizing as the solid phase a MTP.

12. The high resolution method of prostate cancer of claim 11, wherein the assay is fluorescence-based.

13. The high resolution method of prostate cancer of claim 8, wherein the assay is fluorescence-based.

14. The high resolution method of prostate cancer of claim 8, wherein the assay is based on enzyme-generated signal.

15. (canceled)
